(19) United States
(12) Patent Application Publication
(10) Pub. No.: US 2022/0211084 A1
(43) Pub. Date: Jul. 7, 2022

(54) METHOD OF PRODUCING RED SEAWEED SOURCED FOOD INGREDIENT AND PRODUCT OBTAINED BY THE METHOD

(71) Applicant: DUPONT NUTRITION USA, INC., WILMINGTON, DE (US)

(72) Inventors: Erman BIGIKOCIN, West Midlands (GB); Albert TSAI, NEW CASTLE, DE (US); Khamfa PHONCHAREON, BALA CYNWYD, PA (US)

(21) Appl. No.: 17/611,664

(22) PCT Filed: May 20, 2020
(86) PCT No.: PCT/US20/33805

§ 371 Date: Nov. 16, 2021

Related U.S. Application Data
(60) Provisional application No. 62/852,577, filed on May 24, 2019.

(30) Foreign Application Priority Data
Oct. 1, 2019 (EP) 19200802.7

(51) Int. Cl. A23L 17/60; A23L 5/00; A23L 3/40; A23L 5/20; A23L 5/40

(52) U.S. Cl. CPC A23L 17/60; A23L 5/55; A23V 2002/00; A23L 5/21; A23L 5/40; A23L 3/40

(57) ABSTRACT

The present invention relates to seaweed sourced food ingredient with improved functionality and improved sensory profile. The method of the invention contains a rehydrating step of pre-dried seaweed under combined control of temperature and pH, in the presence of a salt solution, in a way that said method does not lead to substantial chemical modification and extraction of phycocolloids in the seaweed, but still provides the food ingredient with improved functionality property and improved sensory profile.

### FIELD

The present invention relates to an industrially feasible method of processing seaweed that provides a food ingredient with functionality property and improved sensory profile.

### BACKGROUND

There is a growing consumer interest for foods and food ingredients that are produced using methods retaining naturalness of the raw material, and in absence of chemically modified components. This consumer approach has been partly responded by the industry by offering reformulated or new products, in which some food additives, particularly preservatives, colors, and flavors have been successfully eliminated, or replaced with more positively recognised alternative ingredients and food additive of natural origin.

However, providing non-chemically modified alternatives to certain food additives such as certain types of modified starches and hydrocolloids has remained to be a challenge due to absence of economically feasible methods to provide new products to deliver unique and desired functionalities (e.g. gelling, viscosity providing, stabilizing) without negatively affecting the sensory properties (e.g. color, odor, taste) of the food systems. Furthermore, said non-chemically modified new products shall also be positively recognised by consumers and regulatory as not chemically modified ingredients when compared to existing additives.

Edible species of seaweeds have been historically consumed by considerable size of populations in food and medicinal preparations for health and wellbeing. For instance, Irish Moss, scientifically named as Chondrus crispus, was used in 19^{th} century for treatment of respiratory health problems in Ireland, and for purposes of nourishing the population, where the extracts of seaweed obtained by boiling was added to thicken and fortify soup broths and beverages. In the 20^{th} century, extraction and processing of seaweed polysaccharides classed as phycocolloids (such as alginates, agar, carrageenans) have gained industrial importance, and today phycocolloids are widely used commercially in the food and pharmaceutical preparations as additives with unique thickening, gelling, and stabilizing functionalities. In this respect, historically it has been practiced by the industrial manufacturers of phycocolloids to use economically preferred methods that are aimed at purifying and maximizing production yields, improving extraction efficiencies, and maximizing the functional performance of provided end-products in the systems of use.

The methods to increase and optimize phycocolloid functionality by processing can be achieved by modifying the natively present polysaccharides into preferable chemical forms for the users. Alkaline treatment of Gracilaria species prior to the extraction step in aqueous medium has been identified in 1950s to obtain red seaweed derived agar with desirable firmer gelling properties, and these findings has led to harvesting and processing of these species which has once been considered unsuitable due to low quality agar yields obtained (McHugh, 2003).

Alkaline treatment of aqueous extracted carrageenans from red seaweed species has also been performed by industry, particularly to modify some natively present non-gelling carrageenan types into gelling carrageenan forms, such as the conversion of nu- and mu-carrageenans into iota- and kappa-carrageenans respectively. Promoting the carrageenan conversion by alkaline treatment is considered as a chemical modification. The term gelling carageenan is commonly used for the forms that are able to form gels, while the term non-gelling carrageenan is commonly used for carrageenans that do not form gels but provide viscosity. Examples types for non-gelling carageenan include mu, nu, lambda, theta, and xi.

During the second half of 20^{th} century, an alternative process was developed where the alkali modified carrageenans were not extracted, but kept within the seaweed matrix to develop a lower cost method to deliver improved functionality semi-refined carrageenan products as opposed to existing refined carrageenan products in the market. For example, the European Union legislation no 231/2012 defines processing steps for obtaining the food additive “E407a Processed Eucheuma Seaweed” as “aqueous alkaline (KOH) treatment at high temperature of the strains of seaweeds Eucheuma cottonii and Eucheuma spinosum”. The described processing step under alkaline conditions and elevated temperature provides an industrially desirable chemical modification of the phycocolloid profile that is noticeably different from the native phycocolloid profile of the source raw materials.

In summary, the processes of alkaline chemical modification and/or extraction of phycocolloids have been advantageously practiced in strongly alkaline conditions and at elevated temperatures by the industry to increase the rates of reaction and/or to increase extracted yields of desirable quality for end applications of use. However, despite the recently increasing consumer interest on edible seaweeds as potential sources for health and wellbeing, the use of chemically modified and/or highly purified extracts of seaweeds in food preparations are not usually positively recognised by some consumers. Today, there is a demand for industrially feasible method for providing seaweed ingredients that are advantaged in the market by being minimally processed, by meeting acceptable food hygiene standards, by not being chemically modified, and by showing acceptable hydrocolloid functionality and sensory profile when used in edible preparations.

An example effort to provide non-chemically modified texturizing ingredient to the market is described in U.S. Pat. No. 9,688,778B2 as producing thermally inhibited starch that is advantaged by not being chemically modified, while being able to provide desirable functional performance as demonstrated by viscosity tests in provided examples in the file. U.S. Pat. No. 9,688,778B2 further describes products can be provided with improved color properties, such as with desirably increased whiteness. However, the disclosed process does not relate to objective of the present invention of obtaining seaweed sourced non-chemically modified food ingredients with improved functionality and sensory property.

U.S. Pat. No. 6,893,479B2 describes a process to release sap using fresh seaweeds as raw material. The sap is used as a liquid fertilizer after suitable treatment, whereas the retained residue is dried for use as raw material for either extracting phycocolloids, or alternatively for direct use in certain applications. However. U.S. Pat. No. 6,893,479B2 has no mention of taste or odor properties of the dried residue in food applications. Additionally, there is no disclosure on a process to retain functional performance and/or to improve sensory properties of the residue to be directly used in food applications, as per the object of the present invention.

### BRIEF DESCRIPTION OF THE DRAWING

FIG. 1—Process chart showing the process steps for the used control and several embodiments of the invention, also defined in the examples.

### DETAILED DESCRIPTION

It is the object of the present invention to provide not chemically modified seaweed ingredients with improved functionality and improved sensory property. The seaweed ingredient of the present invention can be defined as seaweed flours or seaweed fiber. It can be used in food applications as alternatives to chemically modified seaweed sourced food additives such as extracted, refined, or semi-refined forms of phycocolloids. Moreover, it can also be used as improved alternatives to commercially available natural dried edible seaweed products (such as Irish Moss powders) that negatively affect taste and odor of the applications and provides limited functionality.

The present invention further relates to an industrially feasible method of processing seaweed that improves the sensory profile. Said method does not lead to substantial modification and extraction of inherent phycocolloids.

Moreover, the present invention relates to use of above-mentioned inventive seaweed ingredients for stabilizing, and/or texturizing, and/or thickening purposes in edible products.

The seaweeds suitable for use in the present invention are commercially known as red seaweeds, taxonomically belonging to the class of Rhodophyta. Furthermore, the suitable red seaweeds for use in the present invention can be used as food sources for humans and as sources for the phycocolloids of carageenan and/or agar types. Examples of suitable seaweeds belong to the genera consisting of Kappaphycus, Eucheuma, Gigartina, Chondrus, Iriadae, Mazzaella, Mastocarpus, Sarcothalia, Hypnea, Furcellaria, Gracilaria, Gelidium, Gelidiella, Pterocladia, Halymenia and Chondracanthus.

The process object of the present invention starts by providing harvested seaweed material as previously described.

In an alternative embodiment of the invention, harvested seaweed is optionally subjected to a post-harvest treatment prior to the drying step. The suitable post-harvest treatments include known methods in prior art that such as washing, preserving treatments, color removal treatments, odor removal treatments.

An example to post-harvest treatment for preservation effects is salting. The methods of salting for preserving purposes are of methods that are known in the prior art and commonly known by person skilled in the art.

Another example to post-harvest treatment method is the sauna-like treatment described by Ali et al. (2017), who demonstrated noticeable level of color fading by subjecting seaweed to sauna-like conditions prior to the drying step. The described sauna-like treatment condition can be achieved by containing seaweed in a closed medium, such as but not limited to by placing the seaweed in a bag. The use of post-harvest treatments for color removal, such as applying sauna-like treatment, can eliminate the need for using chemical bleaching agents to improve color and other sensory properties of the products.

The post-harvest treatments applied on harvested seaweed that causes substantial level of phycocolloid extraction, chemical modification, or loss of functional properties (such as texturizing abilities in food applications) are not preferable for the present invention. Furthermore, the use of chemical bleaching agents for refining the material, improving color appearance, or sensory properties are not desirable for the present invention. Examples to chemical bleaching agents are hydrogen peroxide, sodium chlorite, other salts of chlorite, peroxides, persulfates.

Following the harvest of the fresh seaweed, and optionally applying post-harvest treatments, the seaweed is subjected to drying step at temperature from about 35° C. to about 120° C.

By drying stems it means to submit to water removal from the harvested seaweed material by use of economically feasible means that are known in the art. Water removal from the harvested seaweed is advantageous as this brings improved stability against decay of seaweed components during the periods of storage before and during the transport to the manufacturing facility. It is preferable to dry the seaweed until moisture content below 45% is achieved. It is preferable to dry the seaweed to moisture content of not more than 40% (w/w), and most preferable to dry seaweed to not more than 30% (w/w). The methods of drying include but not limited to sun drying. Sun drying refers to use of solar radiation as the main source of energy to reduce water from the materials.

In an embodiment of the invention, harvested seaweed prior and/or after the drying step can be washed. The washing process refers to exposing surfaces of seaweed to water which can remove matter such as impurities, salt, debris, sand. The water used in the washing step can be optionally chosen as sea water.

Afterwards, the harvested and dried seaweed is subjected to a rehydrating process. Rehydrating process refers to introducing water into the dried seaweed. It is advantageous to perform rehydration process at elevated temperatures. Rehydration at elevated temperatures can be preferable to increase the rate of hydration, increase the level of hydration, to reduce the level of undesirable microorganisms, and to improve sensory properties.

The rehydrating step (c) is performed in presence of a salt solution, in a pH lower than 9.5, and at a temperature between 20° C. and 85° C., preferably at a temperature between 50° C. and 75° C.

Elevated temperatures can promote chemical modification, and/or extraction of inherent phycocolloids which are both undesirable to obtain the seaweed ingredient object of the invention. These undesirable effects are avoided in the present invention by the presence of salt solution, and combined control of temperature and pH.

The presence of at least one type of salt in the solution is advantageous to prevent or limit the extraction and separation of phycocolloids from the seaweed, particularly at elevated temperatures. It is further advantageous to use certain salts that specifically interacts with certain types of phycocolloids to limit their extraction under certain temperature and pH conditions of the rehydration process. The types and levels of salts to control the phycocolloid extraction are known to the person skilled in the art.

The salts used to prepare salt solution in water for the invention include sodium chloride, potassium chloride, calcium chloride, magnesium chloride, salts of carboxylic acids (e.g., citrate salts such as trisodium citrate), salts of sulfuric acid, salts of phosphoric acid, and their mixtures. As evident to the skilled person, the composition of the salt solution and its related benefits may be further improved by comprising other water soluble and/or water miscible ingredients. Examples to water soluble and water miscible ingredients include sugars, alcohols, sugar alcohols, glycols, surface active agents, maltodextrin, and their derivatives, and their combinations.

It is also advantageous to perform rehydration process by placing seaweed into salt solution at pH not more than 9.5. Adjusting the salt solution pH to below 9.5, particularly at elevated temperatures, is essential to improve seaweed sensory properties without substantial modification of the inherent phycocolloids. For instance, carageenan molecules are known to show highest stability against thermal degradation at pH most preferably near 9.0. Adjusting the pH of salt solution can be performed preferably by use of diluted acids and alkaline salts that are commonly used in the industry. In addition to the chemical modification of phycocolloids, highly alkaline conditions such as pH level above 10 are undesirable as they may lead to newly formed components that negatively alter the organoleptic properties of seaweed.

The seaweed rehydration step is followed by removing the seaweed from the salt solution, such as by physical separation of rehydrated seaweed and draining off the excess salt solution.

The separation of the rehydrated seaweed is optionally followed by subjecting to a size reduction protocol. The size reduction protocol provides reducing the volume of the rehydrated seaweed, and/or reducing particle size of the seaweed. Size reduction protocol may include processes that are commonly known by the skilled person in the art, such as processes of chopping, cutting, crushing, pressing, squeezing, homogenizing.

In one embodiment of the invention, there is a step of pressing the rehydrated seaweed to obtain a liquid fraction and a pomace. The liquid fraction refers to the liquid that is extracted from the pressed seaweed.

The term pomace refers to the remaining residue after liquid fraction has been extracted from the rehydrated seaweed.

The process of pressing can be achieved by applying pressure onto the seaweed by methods known in the art. For example, a screw press, or af filter press can be used for the pressing step. Furthermore, industrially and commercially well-known variants of equipment that are used to juice plants, vegetables, and fruits can be advantageously used for this purpose.

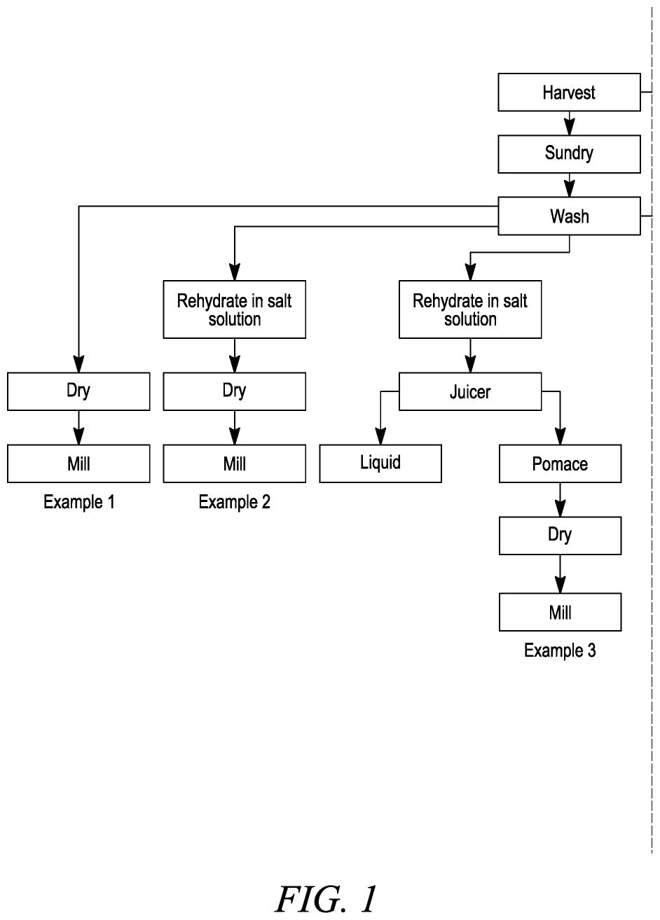

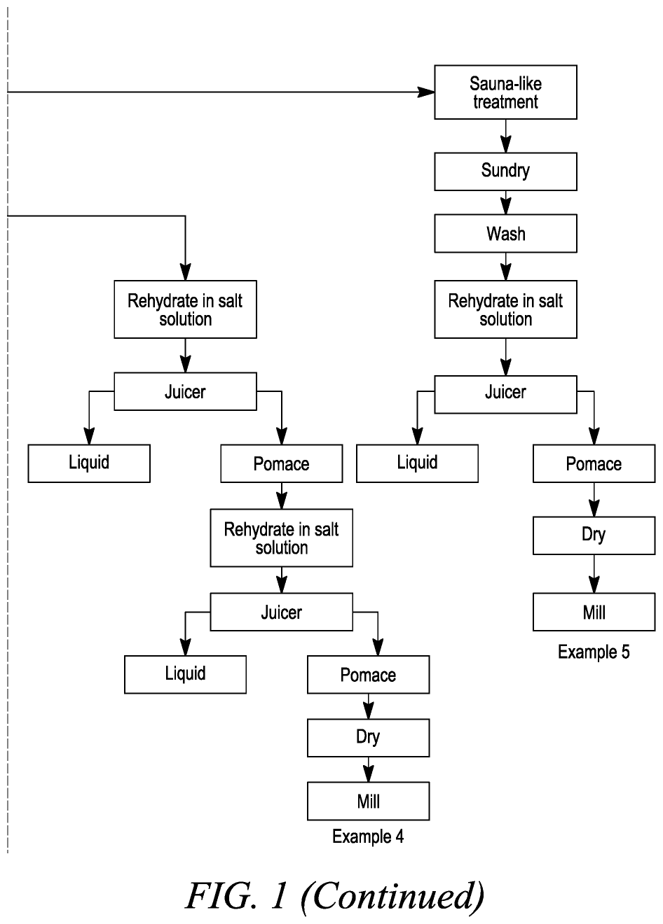

The steps of size reduction and/or pressing can be performed by using a juicer. Certain types of juicers known in the prior art can perform size reduction and pressing simultaneously. Examples types of juicer equipment include but not limited to centrifugal juicers, and masticating juicers. In some of the examples of the invention, as stated in the FIG. 1, the size reduction and pressing steps are indicated as “juicer”.

The rehydrated seaweed directly or optionally submitted to size reduction and/or pressing step is them dried at temperature from about 35° C. to about 120° C. Any drying method can provide moisture content below 15% (w/w) in the product can be suitable. The suitable drying methods include but not limited to belt drying, drum drying, tray drying, tunnel drying, fluid bed drying, and sun drying.

The dried material is then submitted to a milling step. Milling refers to comminution of dried material to obtain fine particles, and it can be achieved by any means known in the prior art. Suitable methods of milling include but not limited to grinding, dry milling, ball milling, jet milling. Preferably the milling method used provides particle size below 250 microns.

The above described process of the invention provides a sensory specific purification of the red seaweed source material. And then the resulting sensory improved red seaweed sourced food ingredient exhibits desirable functionalities (e.g. gelling, viscosity providing, stabilizing) without being a chemically modified product, or a phycocolloid extract.

Some embodiments of this specification are directed to a red seaweed sourced food ingredient made by a method as described in this specification. In some such embodiments, substantially no (e.g., less than 10%) alkaline-driven modification of phycocolloids in the seaweed occurs during the method. In some embodiments, substantially no (e.g., less than 10%) extraction of phycocolloids from the seaweed occurs during the method. In some embodiments, the food ingredient has a reduced odor, taste, and/or color profile that is achieved without use of a bleaching agent during the method. In some embodiments, the food ingredient does not contain any residual chlorite, peroxide, or persulfate compound from an ingredient introduced during the method. In some embodiments, the food ingredient does not contain any residual bleaching agent from the method.

### EXAMPLES

### Test Methods

Viscosity analysis: The viscosity was measured using a Brookfield DVE viscometer using suitable spindle at 30 rpm speed, and reported in centipoises (cP). Samples for viscosity testing were prepared by dispersing 7.5 grams of powder sample in 492.5 grams of deionized water, stirring while heating to 85° C., holding for 10 minutes at 85° C., adding back deionized water (as needed) for 1.5% (w/w) solids, cooling with continuous stirring, and measuring viscosity when equilibrated at 75° C.

Texture analysis in milk gels: Milk gels were prepared by dispersing 42.5 total grams of sample and sucrose weight in 457.5 grams of homogenized whole milk, stirring while heating to 85° C., holding for 10 minutes at 85° C., adding back deionized water (as needed) to target 500 g (net weight) of solution, pouring the hot solution into dishes (70 mm height, 50 mm diameter), and then placing the dishes into a 10° C. water bath for one hour. At the end of one hour, the gels were inverted and placed in test instrument so that the testing plunger will contact the center of the gel. The break force strength (in grams force) and the penetration distance (in millimetres) of the probe are determined using a Texture Analyser TA-XT2i (Stable Micro Systems) with a 21.5 millimeter diameter tapered metal plunger at a descent speed of 1.2 mm/sec.

The amount of sample and sugar dosed into the milk were adjusted to fix the phycocolloid content in the milk gel preparations. For instance, if 2.5 grams of a sample with 66.7% carrageenan content, and 40 grams of sucrose is added to prepare 500 grams of milk gel preparation, then another sample with lower carrageenan content at 59.90% will be dosed higher at 2.784 grams, and sucrose will be dosed at 39.716 grams to prepare 500 grams of milk gel.

Analysis of milk gel preparations are preferable model food systems due to their relevancy to certain suitable food applications where seaweed phycocolloids can be used. For instance, the formed gel texture properties in milk are affected by the specific phycocolloid interactions with the naturally present proteins, minerals, and fats in the milk. Furthermore, such milk gel preparations are particularly suitable for screening sensory properties, such as taste, odor, and color.

Color analysis: Color analysis in CIELab color space was performed using ColorFlex EZ spectrophotometer (Hunterlab, USA) under a D65 light source to determine L*, a*, and b* values. The L* value ranges from 0 (black) to 100 (white), and it was used to determine the lightness or darkness of powder samples. Higher L* value reading of a sample indicates a lighter color appearance.

Odor analysis: Odor analysis was performed using capillary gas chromatography by determining levels of selected odor marker compounds in powder samples. The marker compounds were separated from the product matrix by volatilisation into a headspace by heating in sealed vials. A fixed volume of the headspace is then injected onto an appropriate GLC column. The marker compounds level was quantified by passing the column outlet into a flame ionisation detector where combustion causes a change in potential difference proportional to the marker compound concentration. The marker compounds were identified by their characteristic retention time on the column. Hexanal was selected to be a suitable odor marker compound for odor analysis of seaweed powders, while other suitable compounds can also be identified for this purpose.

Phycocolloid profiling: Total and individual carageenan content analysis by ¹H-NMR spectroscopy. 0.3% (w/w) carageenan samples were prepared by dissolving 3 mg homogenized sample in 1 mL 50 mM KHP buffer (with 0.1% trimethylasilylpropionic acid sodium salt as an internal reference). The samples were heated with agitation for 3 hr at 70° C. NMR measurements were performed at 353K on a 600 MHz Bruker Ascend spectrometer (Bruker Biospins, Rheinstetten, Germany) operating at 14.1 T and equipped with a 5 mm BBO probe (SmartProbe). Standard ¹H NMR spectra were acquired by 64 scans, 64 k data points, spectral width of 20 ppm, an acquisition time of 2.6 s and a recycle time of 20 s. Predefined regions in each spectrum were automatically integrated using a custom made program, and the relative amount of each carageenan type was calculated.

### Tables of Results

TABLE 1
Carrageenan profile of analysed products
Carrageenan profile of analysed samples

| | Lambda | Theta | Nu | Iota | Mu | Kappa | Total |
|---|---|---|---|---|---|---|---|
| Example 1 | 0.6 | 0.0 | 0.6 | 9.1 | 11.7 | 78.0 | 100 |
| Example 2 | 0.9 | 0.0 | 1.1 | 8.7 | 11.8 | 77.5 | 100 |
| Example 3 | 0.8 | 0.0 | 0.8 | 7.9 | 11.9 | 78.6 | 100 |
| Example 4 | 0.5 | 0.0 | 0.3 | 7.4 | 12.2 | 79.6 | 100 |
| Example 5 | 0.0 | 0.0 | 0.6 | 10.8 | 10.8 | 77.8 | 100 |

TABLE 2
Milk gel texture analysis results
Milk gel texture analysis results

| | Carrageenan | Sample | milk gel | milk gel |
|---|---|---|---|---|
| | % in sample | weight (g) | strength (g) | penetration (mm) |
| Example 1 | 66.70 | 2.500 | 83.2 | 5.3 |
| Example 2 | 51.46* | 3.240 | 83.5 | 5.2 |
| Example 3 | 59.90 | 2.784 | 81.5 | 5.3 |
| Example 4 | 61.30 | 2.720 | 78.9 | 5.2 |
| Example 5 | 61.20 | — | — | — |

*calculated maximum carrageenan content by mass balance.

TABLE 3
Detected hexanal and analysed L* values in samples

| | Hexanal (ppb) | L* |
|---|---|---|
| Example 1 | 17510 | 67.0 |
| Example 2 | 9140 | 70.2 |
| Example 3 | 10140 | 69.8 |
| Example 4 | 8480 | 71.5 |
| Example 5 | — | 87.0 |

### Example 1 (Comparative)

Kappaplrycus alvarezii was freshly harvested and dried under sunlight on a clean surface in an open environment for about 3 days until final moisture content of about 24% (w/w) was reached. About 2.0 kg of this sundried seaweed was then washed with water at about 15° C. for 20 minutes to remove surface salt, sand and impurities. The weight of seaweed after the washing step was recorded as 3.1 kg.

450 grams of washed seaweed was then dried on trays placed in a fan assisted oven at 65° C. until reaching constant final dry weight. The dried output from oven was weighed as 125.3 grams, which was then milled for testing purposes.

Analysis by ¹H-NMR spectroscopy indicated about 66.7% total carrageenan content present in the output. The carrageenan profile was composed of 78.0% of kappa, 11.7% of mu, 9.1% of iota, 0.6% of nu, and 0.6% of lambda type, as shown in Table 1.

Texture analysis of milk gel prepared using the milled product provided 83.2 g break strength at 5.3 mm penetration distance, as shown in Table 2.

Odor analysis by capillary gas chromatography detected 17510 ppb of hexanal presence, and the lightness L* value of the milled product was measured as 67.0, as shown in Table 3.

### Example 2

A salt solution composed of 3% (w/w) NaCl, 6% (w/w) KCl, 0.2% (w/w) tri-sodium citrate in deionized water was prepared at room temperature and pH was recorded as 7.2. The salt solution was then heated to 65° C.

450 grams of the washed seaweed from the preparation of Example 1 was placed into the heated salt solution for 1 hour, and the solution temperature was maintained at 65° C. The rehydrated seaweed was separated from the salt solution, drained off from excess solution, and then weighed at about 688 grams. The material was then dried in an oven as described in Example 1. The dried output from oven was weighed as 162.4 grams, which is about 29.6% higher than the dried output obtained in Example 1 preparation. The dried output was then milled for testing purposes.

Analysis by ¹H-NMR spectroscopy indicated the carrageenan profile was composed of 77.5% of kappa, 11.8% of mu, 8.7% of iota, 1.1% of nu, and 0.9% of lambda type, as shown in Table 1.

The similarity of carrageenan profiles of this example and Example 1 indicates no substantial modification of nu- and mu-types of carrageenans into iota- and kappa-types. Furthermore, surprisingly all carrageenan types, regardless of being gelling or non-gelling, have been retained at noticeable levels despite the rehydration at elevated temperature.

Analysis by capillary gas chromatography detected 9140 ppb of hexanal presence as shown in Table 3. The level of hexanal reduction compared to process of Example 1 indicates significant improvement in odor related sensory properties by practicing the inventive seaweed processing method.

The L* value of the milled product was measured at 70.2, which is significantly lighter than the produce of Example 1, as shown in Table 3.

### Example 3

450 grams of the washed seaweed from the preparation of Example 1 was placed into the heated salt solution using the method and salt solution composition described in Example 2. The rehydrated seaweed was separated from the salt solution, drained off from excess solution, and then weighed at about 676 grams. The material was then fed into a masticating juicer to obtain a liquid fraction and a pomace. The obtained pomace was then dried in an oven as described in Example 1. The dried output from oven was weighed as 139.5 grams, which is about 11.3% higher than the dried output of Example 1, and lower than the dried output of Example 2. This illustrates the effect of liquid separation by juicing step on reducing the salt content in the final product. The dried output was then milled for testing purposes.

Analysis by ¹H-NMR spectroscopy indicated about 59.9% total carrageenan content present in the output, and the carrageenan profile was composed of 78.6% of kappa, 11.9% of mu, 7.9% of iota, 0.8% of nu, and 0.8% of lambda type, as shown in Table 1.

The similarity of carrageenan profiles of this example with Example 1 and 2 indicates no substantial modification of nu- and mu-types of carageenan into iota- and kappa-types. Furthermore, surprisingly all carageenan types, regardless of being gelling or non-gelling, have been retained at noticeable levels despite the rehydration at elevated temperature, and obtaining a pomace by separating a liquid fraction.

Furthermore, compared to Example 1, the 11.3% increase in weight and the 59.9% carageenan content indicates no substantial loss of carrageenan after following the steps of rehydration at elevated temperature and the liquid separation to obtain a pomace.

Texture analysis of milk gel prepared using the milled product provided 81.5 g break strength at 5.3 mm penetration distance, as shown in Table 2. Compared to the texture analysis in Example 1, these data show desirable carrageenan functionality has been retained following the steps of rehydration at elevated temperature and the liquid separation to obtain a pomace.

Analysis by capillary gas chromatography detected 10140 ppb of hexanal presence. The level of hexanal reduction compared to process of Example 1 indicates significant improvement in odor related sensory properties by practicing the inventive seaweed processing method.

The L* value of the milled product was measured at 71.5, which is significantly lighter than the produce of Example 1, as shown in Table 3.

### Example 4

450 grams of the washed seaweed from the preparation of Example 1 was placed into the heated salt solution using the method and salt solution composition described in Example 2. The rehydrated seaweed was separated from the salt solution, drained off from excess solution, and then weighed at about 683 grams. The material was then fed into a masticating juicer to obtain a liquid fraction and a pomace.

The obtained pomace was then placed into the heated salt solution using the method and salt solution composition described in Example 2. The rehydrated pomace was separated from the salt solution, drained off from excess solution using sieve, and then weighed at about 804 grams.

The obtained output pomace was then dried in an oven as described in Example 1. The dried output from oven was weighed at about 132.1 grams, which is about 5.43% higher than the dry output of Example 1, and lower than the dried output of Example 3. The dry output was then milled for testing purposes.

Analysis by ¹H-NMR spectroscopy indicated about 61.3% total carrageenan content present in the output, where the carrageenan profile was composed of 79.6% of kappa, 12.2% of mu, 7.4% of iota, 0.3% of nu, and 0.5% of lambda type, as shown in Table 1.

Texture analysis of milk gel prepared using the milled product provided 78.9 g break strength at 5.2 mm penetration distance, as shown in Table 2. Compared to the texture analysis in Example 1, these data show desirable carageenan functionality has been retained following the repeated steps of rehydration at elevated temperature and the liquid separation to obtain a pomace.

Odor analysis by capillary gas chromatography detected 8480 ppb of hexanal presence, as shown in Table 3.

The lightness L* value of the milled product was measured as 71.5, which is significantly lighter than the produce of Example 1, as shown in Table 3.

The further reduction of hexanal level and increase in lightness (L*) value compared to process of Example 2, and Example 3 indicate additional improvement in sensory properties by practicing the inventive seaweed processing method.

### Example 5

Kappaphycus alvrezii was harvested, placed in transparent plastic bags, and stored under sunlight for about 2 hours to create a sauna-like condition. At the end of 2 hours, the bags were rotated and further stored under sunlight for additional 2 hours.

At the end of this sauna-like treatment process, the dark red-brown color appearance of the seaweed was faded into bright-yellow color appearance. The color faded seaweed was taken out of the bags and dried under sunlight on a clean surface in an open environment for about 3 days. The sun-dried seaweed was then washed with water at about 15° C. for 20 minutes to remove surface salt, sand and impurities.

This output material was then fed into a masticating juicer to obtain a liquid fraction and a pomace. The obtained pomace was then dried in an oven as described in Example 1. The washed seaweed was then dried on trays placed in a fan assisted oven at 65° C. until reaching constant final dry weight. The dried output from oven was then milled for testing purposes.

Analysis by ¹H-NMR spectroscopy indicated about 61.2% total carrageenan content present in the output, where the carrageenan profile was composed of 77.8% of kappa, 10.8% of mu, 10.8% of iota, and 0.6% of nu type, as shown in Table 1.

As shown in Table 3, the L* value of the milled product was measured as 87.0, which has an acceptable level of lightness, and creamy white appearance comparable to some refined food additive classified hydrocolloids.

This result show that further improved color appearance can be achieved without use of chemical bleaching agents, and without substantial loss or change in the carrageen profile.

### Example 6 (Comparative)

Kappaphycus striatum var. sacol (green) was harvested and split into two parts. The first part of the harvested seaweed was dried in a fan assisted food dehydrator at 65° C. for about 2 days. The second part of the harvested seaweed was fed into a masticating juicer to obtain seaweed sap fraction and pomace. The obtained pomace was then dried in the fan assisted food dehydrator at 65° C. until the end of water removal. The dried outputs from the two parts were then milled separately for testing purposes.

The viscosity analysis of the milled seaweed of first part, and the milled pomace of second part were measured at 448 cP and 256 cP, respectively. These results indicate significant functionality loss by the process of juicing the harvested seaweed prior to the drying step.

1. A method for the preparation of a red seaweed sourced food ingredient comprising:
a. providing harvested red seaweed,
b. drying the seaweed of step (a),
c. rehydrating the dried seaweed at a temperature of from 20° C. to 85° C. in the presence of a salt solution at a pH that is lower than 9.5,
d. separating the rehydrated seaweed of step (c) from the solution;
e. drying the product of step (d), and
f. milling the dried product of step (e) to form a food ingredient.

2. The method of claim 1, wherein the seaweed is washed between step (a) and step (c).

3. The method of claim 1, wherein in step (b) the seaweed is sundried.

4. The method of claim 1, wherein the drying steps (b) and (e) are performed at a temperature of from about 35° C. to about 120° C.

5. The method of claim 1, wherein the rehydrating step (c) is performed at a temperature of from about 50° C. to about 75° C.

6. The method of claim 1, wherein the salt solution used in step (c) comprises one or more salts selected from the group consisting of sodium chloride, potassium chloride, calcium chloride, magnesium chloride, salts of carboxylic acids, salts of sulfuric acid, and salts of phosphoric acid.

7. The method of claim 1, wherein after step (d) the rehydrated seaweed is optionally subjected to a size reduction protocol.

8. The method of claim 1, wherein before step (e) there is a step of pressing the rehydrated seaweed to obtain a liquid fraction and a pomace.

9. The method of claim 1, wherein the material obtained in step (e) is dried to contain about 15% (w/w) moisture or less.

10. The method of claim 1, wherein at step (f) the dried product of step (e) is grinded to have an average particle size of less than 250 microns.

11. The method of claim 1, wherein the seaweed is selected from the genera consisting of Kappaphycus, Eucheuma, Gigartina, Chondrus, Iriadae, Mazzaella, Mastocarpus, Sarcothalia, Hypnea, Furcellaria, Gracilaria, Gelidium, Gelidiella, Pterocladia, Halymenia and Chondracanthus.

12. The method of claim 1, wherein before drying step (b) the seaweed is submitted to a post-harvest treatment for color and/or odor removal, wherein the post-harvest treatment preferably comprises a sauna-like treatment.

13. The method of claim 1, wherein no bleaching agent, no peroxide, chlorite and/or persulfate compound is introduced to the seaweed during the method.

14. The method of claim 1, wherein substantially no (e.g., less than 100%) alkaline-driven modification and/or extraction of phycocolloids in the seaweed occurs during the method.

15. Red seaweed sourced food ingredient made by the method of claim 1, wherein substantially no alkaline-driven modification and/or extraction of phycocolloids in the seaweed occurs during the method.

16. The red seaweed sourced food ingredient of claim 15, wherein the food ingredient has a reduced odor, taste, and/or color profile that is achieved without use of a bleaching agent.

17. The red seaweed sourced food ingredient of claim 15, wherein the food ingredient does not contain any residual bleaching agent, chlorite, peroxide, or persulfate compound from an ingredient introduced during the method.
